(19) United States
(12) Patent Application Publication
(10) Pub. No.: US 2023/0149195 A1
(43) Pub. Date: May 18, 2023

(54) DEVICES, SYSTEMS, AND METHODS FOR PYLORIC OCCLUSION

(71) Applicant: Boston Scientific Scimed, Inc., Maple Grove, MN (US)

(72) Inventors: Martyn G. Folan, Loughrea (IE); John T. Favreau, Shrewsbury, MA (US); John O'Driscoll, Galway (IE); Thomas M. Keating, Galway (IE)

(73) Assignee: BOSTON SCIENTIFIC SCIMED, INC., Maple Grove, MN (US)

(21) Appl. No.: 17/986,679

(22) Filed: Nov. 14, 2022

Related U.S. Application Data
(60) Provisional application No. 63/279,467, filed on Nov. 15, 2021.

(51) Int. Cl. A61F 5/00

(52) U.S. Cl. CPC A61F 5/0079

(57) ABSTRACT

An occlusion device including a first component configured to anchor the occlusion device with respect to a deployment site, and a second component configured to occlude a lumen through the occlusion device. The first component may be in the form of a stent. The second component may be in the form of a stent or in the form of a liner within the first component. The second component includes an occlusion structure configured to extend across (e.g., transverse to the longitudinal axis of) the lumen of the first component. The occlusion structure may be on a retention member of the second component which engages with a retention member of the first component. Additionally or alternatively, the occlusion structure may be a portion of a liner cinched to occlude the lumen through the occlusion device.

### CROSS-REFERENCE TO RELATED APPLICATIONS

This application claims the benefit of priority of U.S. Provisional Application No. 63/279,467, filed Nov. 15, 2021, the entire disclosure of which is hereby incorporated by reference herein for all purposes.

### FIELD

The present disclosure relates generally to the field of devices, systems, and methods for occluding a body passage, such as the pylorus. More particularly, the present disclosure relates to devices, systems, and methods for occluding a body passage and for reducing migration of an occlusion device with respect to the body passage in which the device is deployed.

### BACKGROUND

Currently there exists a category of medical devices within the gastrointestinal (GI) discipline known as obstruction devices. These devices are designed for obstructing or reducing flow through a body lumen, in particular for obstructing or reducing flow of gastric contents across the pyloric valve (pylorus). Such devices may be known as a pyloric plug or just a “plug”. The plug is particularly useful to stop the flow of stomach contents to the proximal gut (small intestine) which includes the duodenum and the initial part of the jejunum. Such a need arises, for example, after creating an alternative path of flow through a gastro-jejunum or similar anastomosis which bypasses the proximal gut. Other instances in which obstruction or reduction of flow to the proximal gut exist, such as after surgery in the duodenal region or in the pancreas or bile outputs to the duodenum.

Various occlusion devices have become available in the industry. However, there is still a need for improvements to occlusion devices, such pyloric plugs, to resist migration while providing the desired amount of occlusion, and, optionally, allow reversibility of occlusion.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an occlusion device is provided with a primary implantable device defining a lumen therethrough and configured to anchor with respect to tissue of a body passage; a secondary implantable device operatively engaged with the primary implantable device; and an occlusion structure operatively associated with the secondary implantable device and extending across and occluding the lumen of the primary implantable device.

In some embodiments, the primary implantable device is a stent with at least a portion thereof configured to promote tissue ingrowth. In some embodiments, the secondary implantable device is a stent extending through at least a part of the lumen of the primary implantable device; the primary implantable device has a saddle region with a proximal end and a distal end, and at least one retention member extending radially outwardly from the proximal end of the saddle region; and the secondary implantable device has a proximal retention member configured to operatively engage with a proximal retention member of the primary implantable device to be anchored with respect to the body passage. In some embodiments, the occlusion device is a material capable of preventing flow of materials therethrough and is operatively associated with the proximal retention member of the secondary implantable device to extend across the lumen of the primary implantable device. In some embodiments, the proximal retention member of the secondary implantable device fits within a retention space defined between walls of a proximal retention member of the primary implantable device. In some embodiments, the proximal retention member of the secondary implantable device fits over an exterior portion of a proximal retention member of the primary implantable device. In some embodiments, the primary implantable device has at least one retention member extending from each end of the saddle region; and the secondary implantable device has a saddle region with a proximal end and a distal end, the proximal secondary implantable device retention member extending from the proximal end of the saddle region, and the secondary implantable device further including a distal retention member extending from the distal end of the secondary implantable device saddle region. In some embodiments, the secondary implantable device is a liner formed within the lumen of the primary implantable device and shiftable into an occlusion configuration defining the occlusion structure across the lumen of the primary implantable device.

In some embodiments, the occlusion device includes an occlusive material capable of preventing flow of materials therethrough.

In some embodiments, the secondary implantable device is selectively removable from the primary implantable device.

In accordance with various principles of the present disclosure, a method of forming an occlusion device includes engaging a secondary implantable device within a lumen of a primary implantable device configured to anchor with respect to tissue at a deployment site; and extending an occlusion structure of the secondary implantable device across the lumen of the primary implantable device to occlude flow therethrough.

In some aspect of the method, the primary implantable device is a stent with uncoated regions configured to encourage tissue ingrowth, the method further including deploying the primary implantable device and allowing tissue ingrowth with respect to the primary implantable device before engaging the secondary implantable device within the lumen of the primary implantable device.

In some aspects of the method, the occlusion structure includes a material inhibiting tissue ingrowth, the method further including extending the occlusion structure in a direction transverse to the longitudinal axis of the primary implantable device to occlude the lumen thereof. In some aspects, the primary implantable device is a stent with a saddle region and at least one retention member extending radially outwardly from the saddle region and transverse to the longitudinal axis of the primary implantable device, the method further including engaging a retention member of the secondary implantable device with a retention member of the primary implantable device to extend an occlusion structure operatively associated with the retention member of the secondary implantable device across the lumen of the primary implantable device. In some aspects, the primary implantable device is a stent with a saddle region defining the primary implantable device lumen therethrough, and the secondary implantable device includes a liner extending through the primary implantable device lumen, the method further including extending at least a portion of the liner across the primary implantable device lumen to occlude the primary implantable device lumen.

In accordance with various principles of the present disclosure, a method of occluding a body passage includes extending a primary implantable device across the body passage to anchor with respect to tissue of the body passage; securing a secondary implantable device with respect to the primary implantable device, and extending an occlusion device operatively associated with the secondary implantable device across a lumen extending through the primary implantable device to occlude the lumen.

In some aspects, the method includes securing the secondary implantable device with respect to the primary implantable device after sufficient time has elapsed to allow the primary implantable device to be anchored with respect to the tissue of the body passage. In some aspects, the sufficient time allows for tissue ingrowth with respect to the primary implantable device to anchor the primary implantable device with respect to the body passage.

In some aspects, securing the secondary implantable device with respect to the primary implantable device includes operatively engaging a retention member of the secondary implantable device with a corresponding retention member of the primary implantable device.

In some aspects, extending the occlusion device includes shifting a secondary implantable device in the form of a liner within the lumen of the primary implantable device into an occlusion configuration.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a schematic representation of a portion of a gastrointestinal system with an occlusion device, such as formed in accordance with various principles of the present disclosure, positioned across a pylorus.

FIG. 2 illustrates an elevational view, in cross-section, of an embodiment of an occlusion device formed in accordance with various principles of the present disclosure.

FIG. 3 illustrates an elevational view, in cross-section, of another embodiment of an occlusion device formed in accordance with various principles of the present disclosure.

FIG. 4 illustrates an elevational view, in cross-section, of another embodiment of an occlusion device formed in accordance with various principles of the present disclosure.

FIG. 5 illustrates an elevational view, in cross-section, of another embodiment of an occlusion device formed in accordance with various principles of the present disclosure.

FIG. 6 illustrates an elevational view, in cross-section, of another embodiment of an occlusion device formed in accordance with various principles of the present disclosure.

FIG. 7 illustrates an elevational view, in cross-section, of another embodiment of an occlusion device formed in accordance with various principles of the present disclosure.

FIG. 8 illustrates an elevational view, in cross-section, of another embodiment of an occlusion device formed in accordance with various principles of the present disclosure.

FIG. 9A illustrates an elevational view, in cross-section, of another embodiment of an occlusion device formed in accordance with various principles of the present disclosure in a first configuration.

FIG. B illustrates an elevational view, in cross-section, of another embodiment of an occlusion device formed in accordance with various principles of the present disclosure in a second configuration occluding the lumen through the occlusion device.

FIG. 10 illustrates a detail view of a first embodiment of a mechanism for shifting an occlusion device as in FIG. 9A into an occluding second configuration as in FIG. 9B in accordance with various principles of the present disclosure.

FIG. 11 illustrates a detail view of a first embodiment of a mechanism for shifting an occlusion device as in FIG. 9A into an occluding second configuration as in FIG. 9B in accordance with various principles of the present disclosure.

FIG. 12 illustrates an elevational view, in cross-section, of an embodiment of an occlusion device as in FIG. 9B with an instrument inserted therein to release the occluding component thereof.

FIG. 13 illustrates an elevational view of an anchoring component of an occlusion device formed in accordance with various principles of the present disclosure with a removal device deployed therein.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery) , and “distal” refers to the direction or location furthest from the user, such as when using the device (e.g., introducing the device into a patient, or during implantation, positioning, or delivery), and/or closest to a delivery device or treatment site. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, “channel”, “cavity”, “bore”, etc., are not limited to a circular cross-section. As used herein, a “free end” of an element is a terminal end at which such element does not extend beyond.

In accordance with various principles of the present disclosure, an occlusion device is configured to be delivered transluminally (e.g., transcatheterally, etc., such as through potentially tortuous body passages rather than by open surgery) to a deployment site and deployed at the deployment site to occlude passage of materials through or across the deployment site. It will be appreciated that reference may be made herein to terms such as occlude, obstruct, block, plug, and other grammatical forms thereof, such terms being used interchangeably herein without intent to limit, and including full or partial occlusion, etc. It will further be appreciated that terms such as deploy, extend, place, etc., and various grammatical forms thereof, may be used interchangeably herein without intent to limit, with respect to a device as disclosed herein. Moreover, reference may be made to placement of a device through, across, in, etc., such terms being used interchangeably herein without intent to limit. Finally, it will be appreciated that terms such as lumen, passage, etc., may be used interchangeably herein without intent to limit. The deployment site generally is a body passage through which materials normally pass. It will be appreciated that as referenced herein, “materials” which may pass through the lumen of an occlusion device formed in accordance with various principles of the present disclosure include, without limitation, fluids, particles (e.g., chyme), tools, instruments, devices, etc. It may be desirable (e.g., medically indicated) to inhibit or prevent passage of such materials through the body passage, such as selectively and/or reversibly. For instance, it may be desirable to occlude the body passage for a selected period of time, and then to reverse or reduce such occlusion after such selected period of time. In some embodiments, the body passage is a pylorus.

In some embodiments, the occlusion device is delivered with the assistance of a delivery device such as a flexible elongate member (e.g., catheter, endoscope with a working channel therethrough for delivery of devices, etc.). For instance, the occlusion device may be delivered through a working channel of an endoscope (referenced herein as simply a scope for the sake of convenience and without intent to limit) with a catheter-based delivery system by direct vision and/or fluoroscopy. In some embodiments, the occlusion device may be delivered independently of a scope using fluoroscopy. Additionally or alternatively, the occlusion device may be delivered over a guide wire. The occlusion device (or at least the support structure thereof) may be delivered in a delivery configuration configured to facilitate delivery to a treatment site, and shiftable into a deployment configuration, configured to facilitate deployment at a deployment site. In the delivery configuration, the occlusion device may be in a collapsed and/or unexpanded configuration within a lumen of the delivery device. In the deployed configuration, the occlusion device may be in an expanded configuration, such as to be securely deployed at the deployment site. In some embodiments, when in the delivery configuration, the occlusion device (or at least the support structure thereof) is contracted in diameter (e.g., to fit within the delivery device) and extended in length. In some embodiments, when in the delivery configuration, the occlusion device contacts the tissue at the deployment site to be securely positioned with respect to the tissue.

In accordance with various principles of the present disclosure, an occlusion device includes a primary implantable device and a secondary implantable device. The primary implantable device may be considered an anchor or scaffold, with a primary function of securing the occlusion device in place at the deployment site. The secondary implantable device may be considered an occluding device and/or a plug component with a primary function of occluding the lumen through the occlusion device. Reference will be made herein to a “primary” implantable device and a “secondary” implantable device for the sake of convenience and without intent to limit, use of the terms “primary” and “secondary” referring to temporal sequence of deployment (the primary implantable device being deployed before the secondary implantable device is deployed) rather than to priority or importance or other type of ranking or comparison.

The primary implantable device and the secondary implantable device of an occlusion device formed in accordance with various principles of the present disclosure may be in the form of a scaffolding structure, referenced herein, for the sake of convenience and without intent to limit, as a scaffold and/or a stent. In some embodiments, the stent is configured to be placed in a body passage and has a lumen therethrough. The stent may be formed of a metal, a polymer, or a combination thereof. The stent may be configured to shift between a delivery configuration in which the stent is configured (e.g., collapsed, constrained, unexpanded, etc.) to facilitate delivery through a body passage, and a deployed configuration (e.g., expanded) in which the stent is configured to facilitate deployment at a deployment site, such as by contacting the walls of the body passage in which the stent is placed. The stent may be self-expanding, or may be expanded with the assistance of another device (e.g., an expandable balloon). Examples of stents include, without limitation, stents having one or more strut members combined and/or arranged to form a rigid and/or semi-rigid stent structure. For example, the strut members may be formed of one or more wires or filaments which are braided, wrapped, intertwined, interwoven, woven, knitted, looped (e.g., bobbinet-style), knotted, or the like to form a scaffold configuration. Alternatively, the stent may be a monolithic structure formed from a cylindrical tubular member, such as a single, cylindrical laser-cut tubular member, in which the remaining portions of the tubular member form the strut members. The strut members of a self-expanding stent may be formed of shape-memory material, such as Nitinol or Elgiloy, so that the stent returns to a pre-shaped expanded configuration from a collapsed configuration. Openings or interstices may be defined between adjacent strut members.

The primary implantable device is configured to anchor the occlusion device with respect to the deployment site. In accordance with various principles of the present disclosure, the primary implantable device is independent of or may act or function independently of the secondary implantable device in anchoring the occlusion device with respect to the deployment site. The primary implantable device may be considered an anchoring device, an anti-migration device, a docking station, etc., of the occlusion device. In embodiments in which at least the primary implantable device is a stent, the walls of the stent may be formed to promote tissue ingrowth (e.g., hyperplastic tissue growth), at least in sufficient regions of the stent to secure the primary implantable device with respect to tissue at the deployment site. For instance, various regions of the primary implantable device (e.g., portions of the walls of the primary implantable device) may include tissue ingrowth promoting regions, such as uncovered and/or bare metal portions exposed to the tissue at the deployment site. As may be appreciated by those of ordinary skill in the art, a scaffolding structure in the form of a stent may have walls with interstitial spaces into which tissue may grow, thereby securing the support structure at the deployment site. It will be appreciated that other configurations of anti-migration features or structures are within the scope and spirit of the present disclosure. For instance, mechanical structures (e.g., anchors, barbs, teeth, sutures, fins, etc.) promoting implantation of the primary implantable device to resist migration of the occlusion device with respect to the deployment site are within the scope and spirit of the present disclosure. Additionally or alternatively, various regions of the primary implantable device may include tissue ingrowth promoting regions, such as uncovered and/or bare metal portions exposed to the tissue at the deployment site. As may be appreciated by those of ordinary skill in the art, a support structure in the form of a stent may have interstitial spaces in which tissue may grow, thereby securing the support structure at the deployment site.

Examples of embodiments of configurations of stents which may be used to advantage as a primary implantable device of an occlusion device formed in accordance with various principles of the present disclosure include stents such as disclosed in one or more of the following published patent applications, each of which is incorporated by reference herein in its entirety for all purposes: U.S. Patent Application Publication US2009/0281557, filed Apr. 21, 2009, titled Tissue Anchor For Securing Tissue Layers, and published Nov. 12, 2009; U.S. Patent Application Publication US2018/0280166, filed Mar. 29, 2018, titled Stents With Dual Tissue-Wall Anchoring Features, and published Oct. 4, 2018; and U.S. Patent Application Publication US2019/0298559, filed Mar. 22, 2019, titled Device, Systems, And Methods For Pyloric Occlusion, and published Oct. 3, 2019.

In accordance with various principles of the present disclosure, the primary implantable device has a lumen therethrough, and the secondary implantable device is deployed into or with respect to the lumen of the primary implantable device. The secondary implantable device is configured to provide a structure which blocks the lumen through the primary implantable device, such structure being referenced herein as an occlusion structure for the sake of convenience and without intent to limit. In some embodiments, the secondary implantable device has a lumen therethrough as well, and the occlusion structure blocks the lumen through the secondary implantable device, such as in addition to blocking the lumen through the primary implantable device. For instance, the occlusion structure may be positioned to extend across the lumen of the primary implantable device and/or the secondary implantable device, such as transverse to a longitudinal axis of the lumen. In some embodiments, the occlusion structure is a liner or coating (e.g., a membrane-like coating) or other material applied transverse to the lumen through the secondary implantable device to inhibit or to prevent flow of materials therethrough. The liner or coating or other material (referenced herein as a liner for the sake of convenience and without intent to limit) generally may be impervious to materials which may otherwise pass through the lumen of the primary implantable device and/or the secondary implantable device. In some embodiments the secondary implantable device is in the form of a stent, optionally similar in structure to the stent of the primary implantable device, other than the addition of an occlusion element. The occlusion structure may be in the form of a liner applied over at least a portion of a secondary implantable device in the form of a stent, such as transverse to the lumen through the secondary implantable device to inhibit or to prevent flow of materials therethrough. In some embodiments, the secondary implantable device is a liner formed or inserted within the primary implantable device (in other words, a liner without a scaffold such as a stent). Such liner optionally is formed with a lumen therethrough which may be closed (e.g., cinched closed) upon or after deployment within the primary implantable device. In other embodiments, an occlusion structure in the form of a liner is extended across the lumen of the primary implantable device, either supported by a scaffolding structure (such as a stent), or as an independently formed wall extending across the lumen of the primary implantable device.

In embodiments in which the secondary implantable device includes an occlusion structure in the form of or including a liner, the liner may be a liner such as known to those of ordinary skill in the art for use in inhibiting tissue ingrowth in stents or other implantable devices. The liner may be formed of any of a variety of biocompatible materials such as a polymeric material, including, without limitation, polyethylene terephthalate (PET), silicone, polyurethane, etc. The liner may be elastomeric or non-elastomeric. Examples of liners or coating materials for support structures which may be used to advantage in an occlusion device formed in accordance with various principles of the present disclosure disclosed in one or more of the following published patent applications and patents, each of which is incorporated by reference herein in its entirety for all purposes: U.S. Patent Application Publication US2017/0071767, filed Sep. 9, 2016, titled Stent With Coated Struts, and published Mar. 16, 2017; U.S. Patent Application Publication US2019/0298559, filed Mar. 22, 2019, titled Devices, Systems, And Methods For Pyloric Occlusion, and published Oct. 3, 2019; and U.S. Pat. No. 10,779,967 to Walsh et al., titled Stents With Dual Tissue-Wall Anchoring Features, and issued on Sep. 22, 2020. As may be further appreciated by those of ordinary skill in the art, the liner or coating may be applied to selected portions of the primary implantable device to affect the interaction of the material of the primary implantable device with adjacent tissue. For instance, in some embodiments, a coating may be applied to at least a portion of the primary implantable device to inhibit or prevent tissue ingrowth with respect to the primary implantable device (such as tissue ingrowth into the structure of the primary implantable device, such as into interstices or openings therein or in a wall thereof), which may facilitate removal of the occlusion device (such as via removal of the primary implantable device) if desired or medically indicated.

In some embodiments, at least one of the primary implantable device or the secondary implantable device includes a saddle region configured to extend through a body passage (and optionally defining a lumen therethrough). In some embodiments, at least one of the primary implantable device or the secondary implantable device includes at least one retention member extending radially outwardly from one or both ends of the device, such as from one or both ends of the saddle region. The retention members may alternately be referenced as flanges without intent to limit. The retention members may be formed integrally with or separately from the saddle region. In some embodiments, one or more of the retention members are double-wall retention members, the double walls of each retention member being adjacent each other (as referenced herein, adjacent and in contact or spaced apart from each other). In some embodiments, more than one retention member may extend radially from one or each end of the saddle region, adjacent each other (in contact or spaced apart from each other). The lumen through the saddle region of one or both of the primary implantable device and the secondary implantable device may extend through one or more of the retention members extending therefrom.

In some embodiments, the secondary implantable device has at least one retention member which engages with a portion of the primary implantable device, such as a retention member of the primary implantable device, to hold the secondary implantable device in place with respect to the primary implantable device. For instance, the retention member of the secondary implantable device may snap into place, engage, interfit, interlock, cover, etc. (such terms and the like, and other grammatical forms thereof, being used interchangeably herein without intent to limit) with a portion of the primary implantable device, such as a retention member thereof. The occlusion structure (blocking flow through the lumen of the primary implantable device and/or the secondary implantable device) may extend on or across the retention member of the secondary implantable device. A saddle region may extend between the one or more retention members of the secondary implantable device such as to maintain the spaced apart relationship (i.e., maintain the spacing) between the retention members. It will be appreciated that reference herein to a location being at, on, from, along, etc., are not intended to be limited to a precise location. For instance, reference to retention members extending from an end of a device is intended to encompass, without limitation, near, adjacent, in the vicinity of, etc., and even spaced apart from yet closer to an end than a middle of a device. It will further be appreciated that with regard to flow through a body passage, and devices within such body passage, proximal refers to the upstream side of the body passage, and distal refers to the downstream side of the body passage. In embodiments in which an occlusion device is anchored with respect to a body passage, such as a pylorus, the first end to be deployed generally is a distal end, and the second end to be deployed generally is the proximal end. Furthermore, to resist flow of materials through the body passage, an occlusion device formed in accordance with various principles of the present disclosure typically would have a retention member with more resistive or anti-migration force along the proximal end thereof, and an occlusion structure on an end of the device typically would be provided at least along the proximal end of the device.

Although principles of the present disclosure may be applied to occlude flow of materials through any of a variety of body passages, various examples of embodiments are described herein as deployed across a pylorus. In such embodiments, at least the proximal gastric end may have greater resistance to migration (e.g., as a result of stiffness, tissue ingrowth, size, position and/or location of the secondary implantable device, etc.) than the distal duodenal end. However, the principles of the present disclosure have broader application than such example of a deployment site.

It will be appreciated that in accordance with various principles of the present disclosure, occlusion by the occlusion structure may be removable/reversible. For instance, the secondary implantable device and associated occlusion structure may be configured for removal from the primary implantable device. The primary implantable device may also be configured to be removed from the deployment site, with or separately from the secondary implantable device. In some embodiments, one or more of the retention members of the primary implantable device and/or the secondary implantable device may include an extension or lip, generally extending axially away from the saddle region, which may be grasped to facilitate removal of the primary implantable device and/or the secondary implantable device.

Various embodiments of occlusion devices will now be described with reference to examples illustrated in the accompanying drawings. Reference in this specification to “one embodiment,” “an embodiment,” “some embodiments”, “other embodiments”, etc., indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc., in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein, and the examples of embodiments disclosed herein are not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

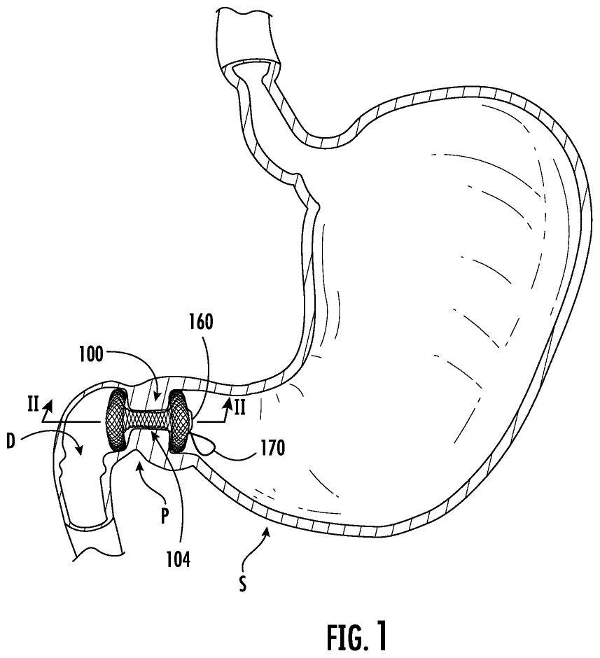

Turning now to the drawings, an example of an embodiment of an occlusion device 100 formed in accordance with various principles of the present disclosure is illustrated in FIG. 1 positioned across a pylorus P as an example of a deployment site for the occlusion device 100. The occlusion device 100 may be configured to shift from a collapsed delivery configuration (not illustrated), in which the occlusion device 100 is sufficiently compact to fit within a flexible elongate member for transluminal delivery to the deployment site, and an expanded deployed configuration, such as illustrated in FIG. 1, in which the occlusion device 100 is securely positioned across a body passage such as a pylorus P.

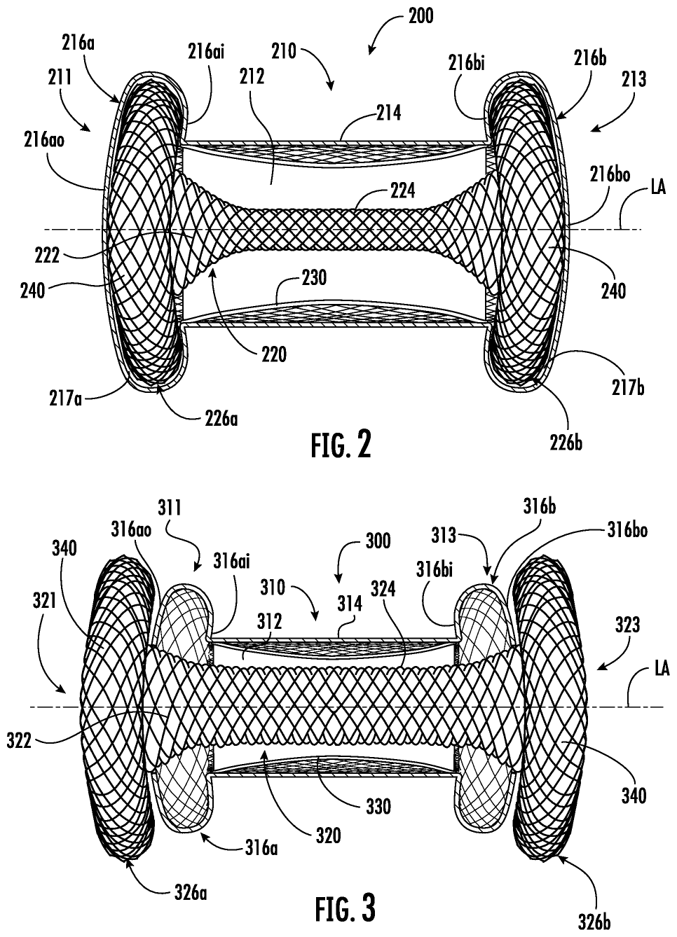

In accordance with various principles of the present disclosure, an occlusion device, such as the occlusion device 100 illustrated in FIG. 1, includes a primary implantable device 210 and a secondary implantable device 220, as illustrated in FIG. 2 (showing a cross-sectional view along line II-II of FIG. 1). In the illustrated example of an embodiment, the primary implantable device 210 and the secondary implantable device 220 are each in the form of a stent configured to shift between a collapsed delivery configuration and an expanded deployed configuration, such as known or heretofore known in the art, the present disclosure not being limited to a specific structure for the stent. The primary implantable device 210 defines a lumen 212 through a saddle region 214 through which the secondary implantable device 220 extends. Additionally, in the example of an embodiment of a primary implantable device 210 illustrated in FIG. 2, at least one retention element 216a extends generally radially outwardly from a first end 211 (e.g., a distal end) of the saddle region 214, and at least one retention element 216b extends generally radially outwardly from a second end 213 (e.g., a proximal end) of the saddle region 214. The retention member 216a extending from the first end 211 of the saddle region 214 may be referenced as a first or distal retention member 216a for the sake of convenience and without intent to limit. The retention member 216b extending from the second end 213 of the saddle region 214 may be referenced as a second or proximal retention member 216b for the sake of convenience and without intent to limit. The retention members 216a, 216b generally extend transverse to (e.g., perpendicular to) a longitudinal axis LA of the occlusion device 200 and/or the primary implantable device 210. Generally, the occlusion device 200, the primary implantable device 210, and the secondary implantable device 220 have a common longitudinal axis LA, though this is not necessarily the case in all embodiments formed in accordance with various principles of the present disclosure. For the sake of convenience, reference is generally made to a longitudinal axis LA, which may be a longitudinal axis of one or more of the occlusion device 200, the primary implantable device 210, and the secondary implantable device 220, without intent to limit to any of these element.

The retention members 216a, 216b may be sized, shaped, configured, and dimensioned to retain the support structure 210 with respect to a deployment site, such as by engaging anatomical structures (e.g., such as walls on either side of the body passage through which the stent extends), such as the duodenum D and the stomach S, respectively, as illustrated in FIG. 1. The retention members 216a, 216b preferably are structured to have sufficient retaining strength to withstand anatomical forces acting to displace the primary implantable device 210, such as in manners known or heretofore known in the art, to thereby dock or anchor the occlusion device 200 in place at the deployment site. In embodiments in which the occlusion device 200 is to be placed across a pylorus P, the saddle region 214 is sized and dimensioned to traverse the pyloric region, and generally has a length of approximately 15-20 mm, and diameter, from stenotic condition (in a stenosed passage) to post-surgical arrangement (e.g., a larger passage) of approximately 10 mm-25 mm. The retention members 216a, 216b generally have a diameter larger than the diameter of the saddle region 214 to promote antimigration from the highly motile pyloric region. In some embodiments, one or both of the retention members 216a, 216b are double-walled retention members, i.e., formed of two walls adjacent each other.

In accordance with various principles of the present disclosure, at least one or more regions of the primary implantable device 210 which contact the tissue at the deployment site are uncoated so that tissue ingrowth into such portion (e.g., wall) of the primary implantable device 210 is promoted. For instance, a portion if not the entire exterior of the saddle region 214 may remain uncoated to promote tissue growth therein. Additionally or alternatively, at least the inwardly facing faces/inner walls 216ai, 216bi (walls if the retention member 216a, 216b are double-wall retention members, or simply faces of a wall if the retention members 216a, 216b are single wall structures), respectively, of the retention members 216a, 216b remain uncoated to promote tissue ingrowth therewith. For the sake of convenience, and without intent to limit, reference is made herein to inwardly-facing walls 216ai, 216bi. In accordance with various principles of the present disclosure, the primary implantable device 210 is deployed before the secondary implantable device 220 is deployed, although simultaneous deployment of the primary implantable device 210 and the secondary implantable device 220 is within the scope of the present disclosure. In some embodiments, the primary implantable device 210 is deployed several days (e.g., 7-10 days) before the secondary implantable device 220 is deployed to allow for tissue ingrowth into at least portions of the primary implantable device 210 so that the primary implantable device 210 may function as an anti-migration component of the occlusion device 200. It will be appreciated that in some embodiments, a liner 230 may be formed spaced inwardly of the saddle region 214 of the primary implantable device 210 to prevent excessive tissue ingrowth. For example, a coating may be formed over a mandrel positioned within the lumen 212 of the primary implantable device 210 to form a liner 230 spaced inwardly from the wall of the saddle region 214 (e.g., forming a loose or baggy liner within the saddle region 214). The liner 230 preferably is composed of a material which inhibits tissue ingrowth, such as any of the materials known in the art for such purpose as discussed above, and optionally is elastic.

In accordance with various principles of the present disclosure, the secondary implantable device 220 is deployed through the lumen 212 of the primary implantable device 210. In the example of an embodiment illustrated in FIG. 2, the secondary implantable device 220 includes a saddle region 224 extending through the saddle region 214 of the primary implantable device 210, and at least one retention member 226a, 226b extending from each end of saddle region 214, respectively. One or both of the retention members 226a, 226b of the secondary implantable device 220 may be a double-walled retention member, the walls of each retention member 226a, 226b being adjacent (as noted above, including, without limitation, contacting each other or spaced apart from each other). The spacing between the double walls of the retention members 216a, 216b of the primary implantable device 210 and the retention members 226a, 226b of the secondary implantable device 220 may be selected to provide an interference fit between the respective retention members 216a, 216b, and 226a, 226b. The secondary implantable device 220 optionally has a lumen 222 defined therethrough as well, though the presence of a lumen is not critical to the principles of the present disclosure as will become evident. In accordance with various principles of the present disclosure, the secondary implantable device 220 is configured to function as a plug component of the occlusion device 200. Additionally, the secondary implantable device 220 preferably is configured to engage the primary implantable device 210 sufficiently securely to avail of the anti-migration properties or features or nature of the primary implantable device 210 such as described above.

A secondary implantable device 220 formed in accordance with various principles of the present disclosure may provide an occlusion structure in any of a variety of manners or shapes or configurations capable of occluding flow of materials through the lumen 202 of the occlusion device 200. For instance, the occlusion structure 240 may be in the form of a liner or coating (as noted above, such terms may be used interchangeably herein without intent to limit) operatively associated with at least a portion of the secondary implantable device 220. It will be appreciated that reference may be made herein to an occlusion structure being operatively associated with, on, coupled to, positioned across, etc., a secondary implantable device, such terms and other grammatical forms thereof being used interchangeably herein without intent to limit. In the example of an embodiment illustrated in FIG. 2, a coating is formed on one or both of the retention members 226a, 226b. If the retention members 226a, 226b are double-walled retention members 226a, 226b, then the coating may be applied to one or both of the double walls of a given retention member 226a, 226b. The coating is composed of a material selected to occlude the flow of material which normally would flow across the body passage in which the occlusion device 200 is deployed. For instance, as applied across a pylorus P, the coating is composed of a material capable of preventing flow of chyme and other food matter therethrough, such as known to those of ordinary skill in the art, examples of which are provided above. In some embodiments, the coating forms a liner wall, extending across the secondary implantable device 220 transverse to the longitudinal axis LA, such as across the lumen 222 of the secondary implantable device 220 and transverse to at least the longitudinal axis LA of the secondary implantable device 220. The size, shape, configuration, and dimensions of the occlusion structure 240 may be selected to achieve the desired degree of occlusion through the lumen 212 of the primary implantable device 210. For instance, the occlusion structure 240 may be sized, shaped, configured, and dimensioned to substantially completely occlude the lumen 212 or may be sized, shaped, configured, and dimensioned to partially occlude the lumen 212. As may be appreciated, the secondary implantable device 220 may be removable, and replaceable with another secondary implantable device 220 with a differently sized, shaped, configured, and dimensioned occlusion structure 240, such as if medically indicated.

As noted above, the secondary implantable device 220 is configured to securely engage the primary implantable device 210, and thereby to be held in place at the deployment site by virtue of the secure engagement of the primary implantable device 210 with tissue at the deployment site, or otherwise via the primary implantable device 210. In the example of an embodiment of an occlusion device 200 illustrated in FIG. 2, the secondary implantable device 220 is configured to engage the primary implantable device 210 via respective retention members 216a, 216b, 226a, 226b. For instance, in the example of an embodiment of a primary implantable device 210 illustrated in FIG. 2, the retention members 216a, 216b are double-wall retention members, such as in the form of a torus, with respective inwardly-facing walls 216ai, 216bi spaced apart from respective outwardly-facing walls 216ao, 216bo, defining respective retention spaces 217a, 217b therebetween. In the example of an embodiment illustrated in FIG. 2, the retention members 226a, 226b of the secondary implantable device 120 are configured to securely engage within the retention spaces 217a, 217b, such as by snapping therein, fitting therein with an interference fit, or otherwise fitting therein to resist migration with respect to the primary implantable device 210. By virtue of the resistance of the primary implantable device 210 to migration with respect to the deployment site, and the engagement of the secondary implantable device 220 with the primary implantable device 210, the secondary implantable device 220 resists migration with respect to the deployment site as well. It will be appreciated that in some embodiments, the saddle region 224 of the secondary implantable device 220 may be considered to serve at least a basic function of maintaining the retention members 226a, 226b of the secondary implantable device 220 at an appropriate distance such that the retention members 226a, 226b of the secondary implantable device 220 securely engage with the retention members 216a, 216b of the primary implantable device 210. In some embodiments, the saddle region 224 imparts tension (e.g., as a result of natural forces of a braided structure to contract) between the retention members 226a, 226b of the secondary implantable device 220 to hold the retention members 226a, 226b in place with respect to the retention members 216a, 216b of the primary implantable device 210. For instance, if a liner 230 is provided within the primary implantable device 210, then the saddle region 224 of the secondary implantable device 220 has a sufficiently low profile (small diameter) as to not interfere with the liner 230. In some embodiments, the retention members 226a, 226b of the secondary implantable device 220 are double-wall retention members similar to the retention members 216a, 216b of the primary implantable device 210.

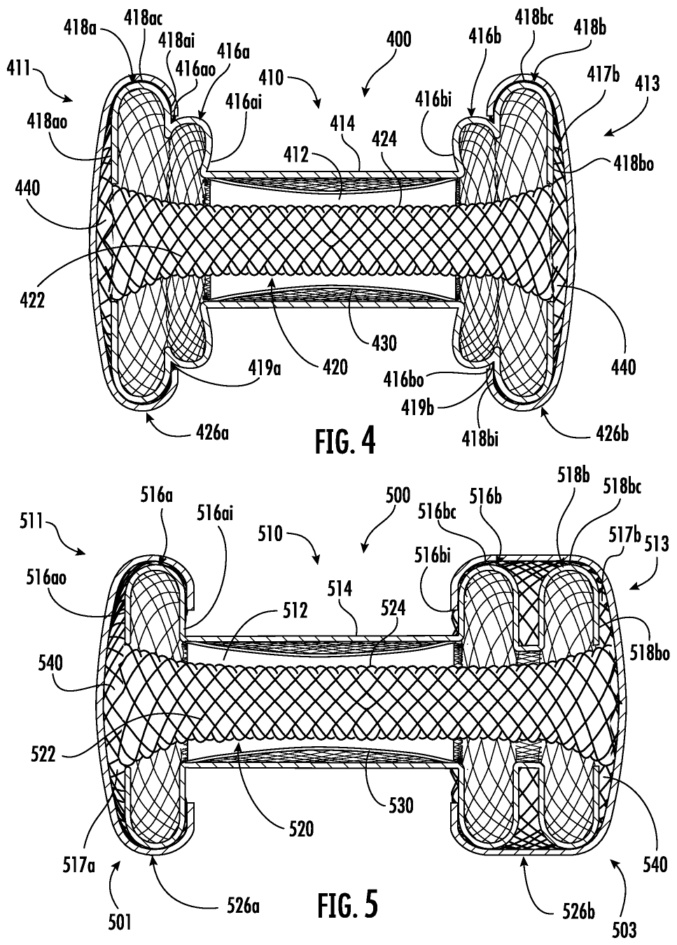

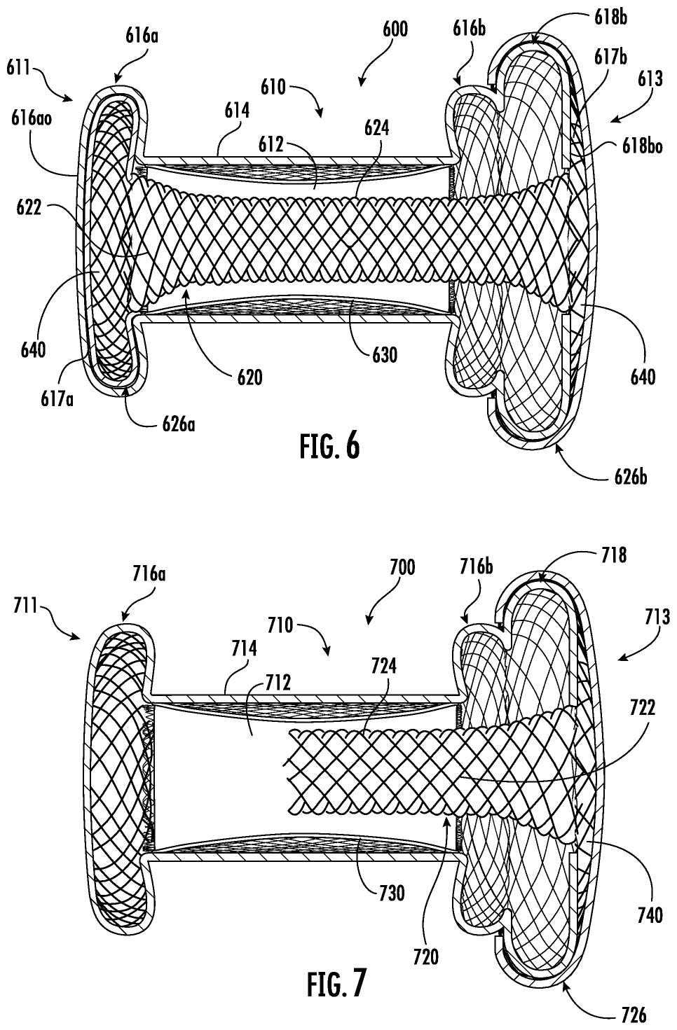

Instead of both retention members of the secondary implantable device fitting within the corresponding retention members of the primary implantable device, as in the example of an embodiment illustrated in FIG. 2, one or both of the retention members of the secondary implantable device may engage the retention members of the primary implantable device in other manners. For instance, at least one retention member of the secondary implantable device may engage an exterior portion of a corresponding retention member of the primary implantable device. The secondary implantable device may be provided with a second retention member engaged within or with an exterior of another, corresponding, retention member of the primary implantable device. Various examples of embodiments of secondary implantable devices with retention members engaging portions of the primary implantable device (e.g., retention members of a primary implantable device) in various manners and configurations are illustrated in FIG. 3, FIG. 4, FIG. 5, FIG. 6, and FIG. 7, as will now be described in further detail.

Turning to FIG. 3, an example of an embodiment of an occlusion device 300 is illustrated with a primary implantable device 310 and a secondary implantable device 320 extending through a lumen 312 of the primary implantable device 310. As in the embodiment illustrated FIG. 2, the primary implantable device 310 is configured to anchor the occlusion device 300 securely with respect to a deployment site, such as via a combination of tissue ingrowth promoting features, anchoring features, the structure and configuration of the retention members 316a, 316b thereof, etc. The retention members 316a, 316b of the primary implantable device 310 may be double-wall retention members, the configuration of the retention members 316a, 316b optionally contributing to the retention capability of the occlusion device 300. Optionally, the primary implantable device 310 is deployed separately and before the secondary implantable device 320 is deployed to allow sufficient time for the primary implantable device 310 to establish secure anchoring with respect to the deployment site. However, it will be appreciated that the primary implantable device 310 and secondary implantable device 320 may, alternatively, be deployed at the same time, either sequentially, or with the secondary implantable device 320 within the primary implantable device 310 during deployment.

The secondary implantable device 320 has retention members 326a, 326b which engage with respective retention members 316a, 316b of the primary implantable device 310. Unlike the retention members 226a, 226b of the secondary implantable device 220 of the example of an embodiment illustrated in FIG. 2, the retention members 326a, 326b of the secondary implantable device 320 of the example of an embodiment illustrated in FIG. 3 engage an exterior portion of respective retention members 316a, 316b of the primary implantable device 310. More particularly, at least one of the retention members 326a, 326b of the secondary implantable device 320 engages at least a portion of the outwardly-facing walls 316ao, 316bo of a corresponding retention member 316a, 316b of the primary implantable device 310. In the illustrated example of an embodiment in FIG. 3, each end 321, 323 of the secondary implantable device 320 extends completely through the lumen 312 of the primary implantable device 310 (extending through the retention members 316a, 316b) and beyond the respective ends 311, 313 of the primary implantable device 310 so that the retention members 326a, 326b of the secondary implantable device 320 extend beyond the retention members 316a, 316b of the primary implantable device 310.

The saddle region 324 of the secondary implantable device 320 of the occlusion device 300 illustrated in FIG. 3 may function not only to maintain spacing between the retention members 326a, 326b of the secondary implantable device 320, but also to create tension on the retention members 326a, 326b of the secondary implantable device 320 to aid in their retention with respect to the retention members 316a, 316b of the primary implantable device 310. More particularly, the saddle region 314 of the secondary implantable device 320 may serve a spring-like function to keep the retention members 326a, 326b of the secondary implantable device 320 in place across the primary implantable device 310, and/or to hold the retention members 316a, 316b of the primary implantable device 310 in place with respect to the deployment site.

As in the previously described example of an embodiment, the secondary implantable device 320 of the occlusion device 300 illustrated in FIG. 3 includes an occlusion structure 340 capable of occluding flow of materials through the lumen 302 of the occlusion device 300. The occlusion structure 340 may be similar to the occlusion structure 240 described above with respect to the occlusion device 200 illustrated in FIG. 2. In the example of an embodiment of an occlusion device 300 illustrated in FIG. 3, the occlusion structure 340 may be positioned across at least a portion of one or both of the retention members 326a, 326b of the secondary implantable device 320 which extends over the opening of the lumen 312 through the primary implantable device 310 at either end 311, 313 thereof. In some embodiments, the occlusion structure 340 extends over the entire outwardly-facing surface of the retention members 326a, 326b of the secondary implantable device 320. As in the above-described embodiment of an occlusion device 200 illustrated in FIG. 2, the retention members 326a, 326b of the secondary implantable device 320 of the occlusion device 300 illustrated in FIG. 3 may be double-wall retention members. The occlusion structure 340 may extend optionally over both walls of a double-wall retention member 326a, 326b.

The retention members 316a, 316b of the primary implantable device 310 of the occlusion device 300 of FIG. 3 may serve as a physical isolator for the retention members 326a, 326b of the secondary implantable device 320 to discourage tissue ingrowth into the retention members 326a, 326b of the secondary implantable device 320. As such, the secondary implantable device 320 may be removable, and optionally replaceable with another secondary implantable device 320 with a differently sized, shaped, configured, and dimensioned occlusion structure 340, such as if medically indicated.

The primary implantable device and/or the secondary implantable device of an occlusion device formed in accordance with various principles of the present disclosure may have various other configurations of retention members which differ in various aspects from those described above, and may be in various combinations and in various positions relative to one another.

In the example of an embodiment of an occlusion device 400 illustrated in FIG. 4, the configurations of the retention members 426a, 426b of the secondary implantable device 420 and optionally also the retention members 416a, 416b of the primary implantable device 410 may be modified relative to those of the aforementioned examples of embodiments. Instead of the retention members 426a, 426b of the secondary implantable device 420 being double-walled retention members similar to the retention members 416a, 416b of the primary implantable device 410, such as in the form of a torus, one or both of the retention members 426a, 426b of the secondary implantable device 420 may extend over the retention members 416a, 416b of the primary implantable device 410, and then extend towards the saddle regions 414, 424 of the primary implantable device 410 and the secondary implantable device 420, respectively. In some embodiments, such configuration of the retention members 426a, 426b of the secondary implantable device 420 may wrap around one or both ends 411, 413 of the primary implantable device 410, covering not only the outwardly-facing walls of the retention members 416a, 416b of the primary implantable device 410, but also extending substantially along the longitudinal axis LA of the occlusion device 400 to extend over peripheral or radial surfaces of the retention members of the primary implantable device 410, and optionally also over at least a portion of the retention members of the primary implantable device 410 facing towards the saddle region 414 of the primary implantable device 410, as described in further detail below. In some embodiments, the retention members 426a, 426b of the secondary implantable device 420 may be considered to form a cap over the retention members 416a, 416b, respectively, of the primary implantable device 410. In some embodiments, the retention members 426a, 426b of the secondary implantable device 420 form a double-wall torus structure in a reverse direction from that of the previously-described embodiments, with the toroid shape opening towards the saddle regions 414, 424 of the primary implantable device 410 and the secondary implantable device 420, respectively. An occlusion structure 440 is operatively associated with one or both of the retention members 426a, 426b of the secondary implantable device 420 in a manner similar to as described above. The occlusion structure 440 may be formed or provided over any openings through the retention members 426a, 426b, such as formed by the lumen 422 through the secondary implantable device 420.

In some embodiments, the primary implantable device may have more than one retention member along one or both ends thereof. The one or more retention members along each end of the primary implantable device may be spaced apart from one another or optionally partially in contact with one another, with a depression therebetween. For instance, one of each of the adjacent retention members may have at least a portion of a wall facing a wall of the other of the adjacent retention members, the walls optionally extending along each other and being spaced apart from or contacting each other (and optionally forming a groove therebetween). For instance, as illustrated in FIG. 4, the primary implantable device 410 includes not only a first retention member 416a along a first end 411 thereof, and a second retention member 416b along a second end 413 thereof, but also at least one additional retention member 418a along the first end 411 and/or at least one additional retention member 418b along the second end 413. For the sake of convenience, and without intent to limit, the retention member closest to the saddle region is referenced as the inner or first retention member, and the retention members sequentially further from the saddle region are referenced as an outer, or second, third, etc., retention members. In some embodiments, two or more retention members on either end of the primary implantable device 410 may be spaced apart from one another. In other embodiments, two or more retention members on either end of the primary implantable device 410 may contact one another. In some embodiments, each of the one or more retention members 416a, 416b, 418a, 418b has a respective inwardly-facing wall 416ai, 416bi, 418ai, 418bi (facing toward the saddle region 414) and a respective outwardly-facing wall 416ao, 416bo, 418ao, 418bo (facing away from the saddle region 414). In the illustrated embodiment, the outwardly-facing wall 416ao, 416bo of the inner retention members 416a, 416b, respectively, and/or the inwardly-facing wall 418ai, 418bi of the outer retention members 418a, 418b, respectively, does not extend the full length of the respective retention members 416a, 416b, 418a, 418b. In other words, the walls 416ao, 416bo, 418ai, 418bi of adjacent retention members 416a, 416b, 418a, 418b do not extend the full distance from the radial surfaces 416ar, 416br, 418ar, 418br thereof to the saddle region 414 of the primary implantable device 410. In such embodiment, the double retention member configuration may be considered a double torus in series with a depression between the tori as the walls of the tori which face each other are adjoined and form a groove 419a, 419b. It will be appreciated that although the first retention members 416a, 416b are shown as having a smaller diameter than the second retention members 418a, 418b, the present disclosure encompasses a reverse configuration of relative diameters, or retention member 416a, 416b, 418a, 418b having substantially the same diameters. It may be advantageous in some instances for the first retention members 416a, 416b to be larger to provide a “cushion” against the surrounding anatomy through which the primary implantable device 410 extends. Alternatively the second retention members 418a, 418b may be larger to provide more volume of scaffolding material and thus less chance of undesired migration and/or removal. For instance, a 20 mm primary implantable device 410 may have at least one retention member 416a, 416b, 418a, 418b with a 40 mm diameter as a “large” retention member. It will be appreciated that larger retention members may be desirable in, for example, a post surgically altered anatomy.

As described above, at least a portion of the one or both of the retention members 426a, 426b of the secondary implantable device 420 may extend over the retention members of the primary implantable device 410. In the example of an embodiment illustrated in FIG. 4, in which a pair of retention members is provided on at least one end of the primary implantable device 410, the one or both of the retention members 426a, 426b of the secondary implantable device 420 extend over at least the outer/second retention members 418a, 418b, respectively, of the primary implantable device 410. More particularly, the one or both of the retention members 426a, 426b of the secondary implantable device 420 may extend over at least the outer walls 418ao, 418bo of the outer retention members 418a, 418b of the primary implantable device 410. Optionally, as described above, the one or both of the retention members 426a, 426b of the secondary implantable device 420 may extend over the circumferential surfaces 418ac, 418bc of the outer retention members 418a, 418b of the primary implantable device 410. The one or both of the retention members 426a, 426b of the secondary implantable device 420 may further respectively extend over at least a portion of the inwardly-facing walls 418ai, 418bi of the outer retention members 418a, 418b respectively, of the primary implantable device 410, and optionally into the respective groove 419a, 419b between the respective pair of retention members 416a, 418a and 416b, 418b.

As may be appreciated, such as in view of the above-described embodiments, at least the inner retention members 416a, 416b of the primary implantable device 410 may serve as a physical isolator for the respective retention members 426a, 426b of the secondary implantable device 420 to discourage tissue ingrowth into the retention members 426a, 426b of the secondary implantable device 420. As such, the secondary implantable device 420 may be removable, and optionally replaceable with another secondary implantable device 420 with a differently sized, shaped, configured, and dimensioned occlusion structure 440, such as if medically indicated.

It will be appreciated that various other configurations of retention members of the primary implantable device and the secondary implantable device, in various combinations, are within the scope and spirit of the present disclosure, the present disclosure not being limited to the examples of embodiments illustrated in the accompanying figures. For instance, if more than one retention member is provided on a primary implantable device, those retention members may have the same or different diameters, some of the retention members may be spaced apart while others are in contact, etc. Additionally or alternatively, various configurations of retention members of a secondary implantable device in various combinations and relationships with retention members of a primary implantable device are within the scope and spirit of the present disclosure. For instance, an external retention member of the secondary implantable device may extend over a single retention member of the primary implantable device without another retention member being provided adjacent, adjoining, etc., such single retention member of the primary implantable device. Additionally or alternatively, an external retention member of the secondary implantable device may extend to varying extents over a retention member of a primary implantable device. Various other configurations are within the scope and spirit of the present disclosure, the present disclosure not being limited by the examples of embodiments illustrated in the accompanying drawings.

An example of an embodiment of an occlusion device 500 with a primary implantable device 510 having different configurations of retention members at different ends of the saddle region 514 thereof is illustrated in FIG. 5. As illustrated, the primary implantable device 510 has a single retention member 516a on one end and a double retention member 516b on the other end. Such embodiment may be configured for deployment across a pylorus P (as illustrated in FIG. 1), with the single retention member 516a on the distal end 501 of the occlusion device 500 configured to be deployed in a duodenum, and the double retention member (with an inner retention member 516b and an outer retention member 518b) on the proximal end 503 of the occlusion device 500 configured to be deployed in a stomach. Although the retention members 516b, 518b of the double retention member may be in contact with each other (such as in the example of an embodiment illustrated in FIG. 4), in the example of an embodiment illustrated in FIG. 5 the retention members 516a, 518b of the double retention member are spaced apart from each other. Although the retention members 516a, 518b are illustrated as having the same diameter, the diameters may be different from each other (the diameter of the outer retention member 518b being either larger or smaller than the diameter of the inner retention member 516b).

The external retention member of the secondary implantable device 520 may wrap over a single retention member, or more than one retention member. In the example of an embodiment of an occlusion device 500 illustrated in FIG. 5, the external retention members 526a, 526b of the secondary implantable device 520 wrap over the retention members along the respective ends 511, 513 of the primary implantable device 510. More particularly, the external retention member 526a along the first end 521 of the secondary implantable device 520 wraps over the single retention member 516a at the first end 511 of the primary implantable device 510, and the external retention member 526b along the second end 523 of the secondary implantable device 520 wraps over at least a portion of the outer retention member 518b (and optionally over at least a portion of the inner retention member 516b) along the second end 513 of the primary implantable device 510. If the retention members 516a, 518b of the primary implantable device 510 are spaced apart from each other, such as illustrated, for example, in FIG. 5, the external retention member 526b of the secondary implantable device 520 may wrap around only the outer retention member 518b, and may optionally extend within close proximity to or in contact with the saddle region 514 of the primary implantable device 510, extending over respective circumferential surfaces 516bc, 518bc of the retention members 516bc, 518bc of the retention members 516b, 518b. In some embodiments, the external retention member 526b of the secondary implantable device 520 may also wrap partially or more around the inner retention member 516b of the primary implantable device 510. An occlusion structure 540 is operatively associated with one or both of the retention members 526a, 526b of the secondary implantable device 520 in a manner similar to as described above.

As may be appreciated, such as in view of the above-described embodiments, at least the inner retention members 516a, 516b of the primary implantable device 510 may serve as a physical isolator for the respective retention members 526a, 526b of the secondary implantable device 520 to discourage tissue ingrowth into the retention members 526a, 526b of the secondary implantable device 520. As such, the secondary implantable device 520 may be removable, and optionally replaceable with another secondary implantable device 520 with a differently sized, shaped, configured, and dimensioned occlusion structure 540, such as if medically indicated.

It will further be appreciated that the configuration of the retention members of the secondary implantable device may not be the same on the two ends of the secondary implantable device. For instance, in an example of an embodiment of an occlusion device 600 illustrated in FIG. 6, one retention member 626a of the secondary implantable device 620 may fit within a retention space 617a in a double wall retention member 616a along one end 611 the primary implantable device 610, while an external retention member 626b may wrap over the exterior of at least one of one or more retention member 616b, 618b on the other end 613 of the primary implantable device 610. It will be appreciated that the retention member of the primary implantable device 610 over which the external retention member wraps over may be a single retention member, as in the examples of embodiment illustrated in FIG. 2 and FIG. 3 and FIG. 5, or may include more than one retention member, as in the example of an embodiment illustrated in FIG. 4. An occlusion structure 640 is operatively associated with one or both of the retention members 626a, 626b of the secondary implantable device 620 in a manner similar to as described above.

As may be appreciated, such as in view of the above-described embodiments, at least the inner retention members 616a, 616b of the primary implantable device 610 may serve as a physical isolator for the respective retention members 626a, 626b of the secondary implantable device 620 to discourage tissue ingrowth into the retention members 626a, 626b of the secondary implantable device 620. As such, the secondary implantable device 620 may be removable, and optionally replaceable with another secondary implantable device 620 with a differently sized, shaped, configured, and dimensioned occlusion structure 640, such as if medically indicated.

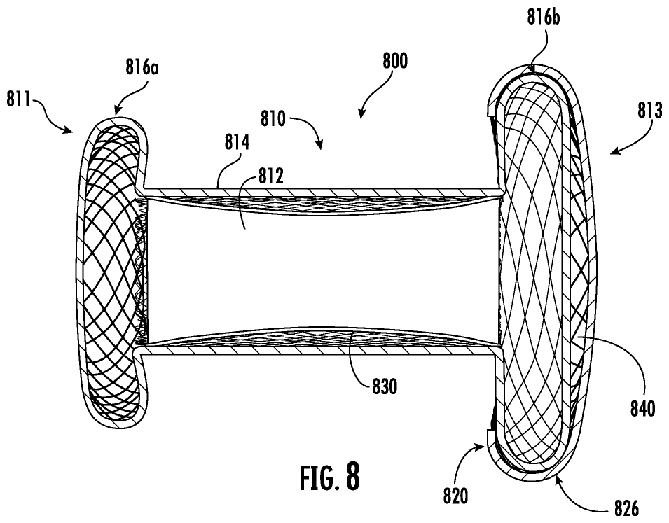

Although the saddle region of the secondary implantable device has been described thus far as spacing apart the retention members at the opposite ends of the secondary implantable device, in some embodiments, the secondary implantable device may have a retention member on only one end thereof. For instance, in the embodiment of FIG. 7, the secondary implantable device 720 has a saddle region 724 with only one retention member 726 with an occlusion structure 740 (similar to the above-described occlusion structures) operatively associated therewith. Retention members 716a, 716b and optional additional outer retention member 718b may extend from the saddle region 714 of the primary implantable device 710 such as described above, the embodiment of a secondary implantable device not being limited by the configuration of the primary implantable device. In an embodiment which is positioned across a pylorus (such as illustrated in FIG. 1), the single retention member 726 may be a proximal retention member 726 on the gastric side of the pylorus, along the stomach. The saddle region 724 may facilitate delivery, deployment, removal, etc., of the secondary implantable device 720. Such saddle region 724 may act to center the single retention member 726 (e.g., on the gastric side of a pylorus) and/or aid in delivery of the secondary implantable device 720. However, as illustrated in the example of an embodiment of an occlusion device 800 of FIG. 8, a secondary implantable device 820 may not include a saddle region, and may simply include a retention member 826 with an occlusion structure 840 (similar to the above-described occlusion structures) configured to occlude the flow of materials through a retention member 816b of the primary implantable device 810. As may be appreciated, such as in view of the above-described embodiments, at least the retention member or members 716b, 718b, 816b of the primary implantable device 710, 810 may serve as a physical isolator for the respective retention members 726a, 826b of the secondary implantable device 720, 820 to discourage tissue ingrowth into the retention members 726a, 826b of the secondary implantable device 720, 820. As such, the secondary implantable device 720, 820 may be removable, and optionally replaceable with another secondary implantable device 720, 820 with a differently sized, shaped, configured, and dimensioned occlusion structure 740, 840 such as if medically indicated.

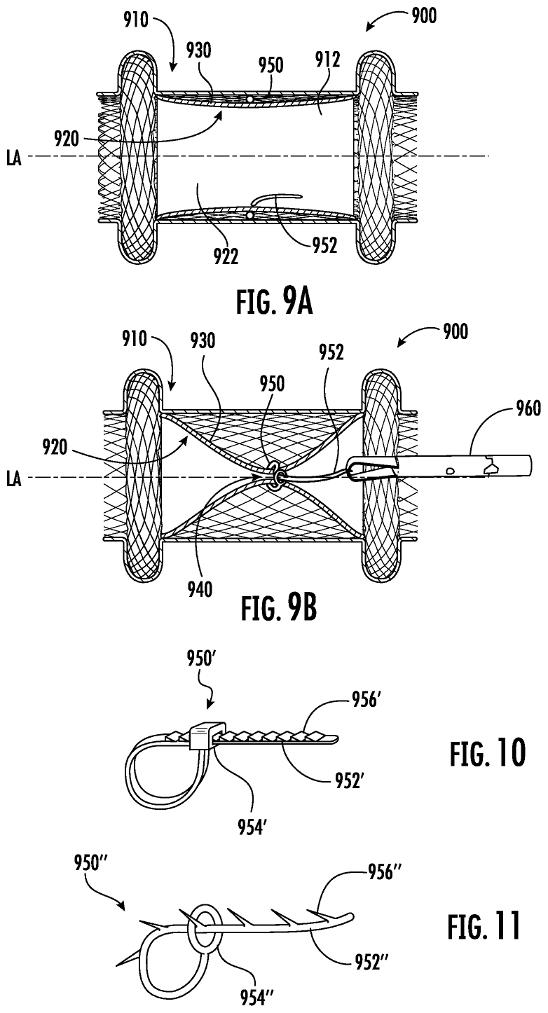

Instead of engaging the retention members of the primary implantable device, as in the examples of embodiments illustrated in FIGS. 2-7, the secondary implantable device may engage the primary implantable device in other manners. For instance, the secondary implantable device may engage one or more structures of the primary implantable device other than the retention members. In the example of an embodiment of an occlusion device 900 illustrated in FIG. 9A and FIG. 9B, a secondary implantable device 920 is in the form of an adjustable liner positioned with the lumen 912 of the primary implantable device 910. In a delivery configuration, such as illustrated in FIG. 9A, the secondary implantable device 920 defines a lumen 922 therethrough. After deployment, such as after the primary implantable device 910 has been allowed sufficient time to be securely implanted with respect to tissue at the deployment site, the secondary implantable device 920 may be shifted into an occlusion configuration, such as illustrated in FIG. 9B. More particularly, the secondary implantable device 920 may be reconfigured to form an occlusion structure 940 extending across (e.g., transverse to the longitudinal axis LA of) the lumen 922 of the secondary implantable device 920, to occlude the lumen 912 of the primary implantable device 910 and/or the lumen 902 of the occlusion device 900, such as in the occluded configuration illustrated in FIG. 9B. In some embodiments, the configuration of the secondary implantable device 920 is adjustable to adjust (either to increase or decrease) the extent to which the occlusion structure 940 occludes the lumen 912 of the primary implantable device 910 and/or the lumen 902 of the occlusion device 900. In some embodiments, the secondary implantable device 920 is adjustable to shift from the initial deployment configuration to an occlusion configuration forming an occlusion structure 940, such as by locking the occlusion structure 940 into a desired occlusion configuration, such as fully closed, such as to remain in the selected occlusion configuration.

The occlusion device 900 illustrated in FIG. 9A and FIG. 9B may include a closure device 950 operatively associated with the secondary implantable device 920 and actuatable to shift the secondary implantable device 920 from the unoccluded configuration illustrated in FIG. 9A to the occluded configuration illustrated in FIG. 9B. The closure device 950 may be in any of a variety of configurations capable of shifting the secondary implantable device 920 into an occlusion configuration. In some embodiments, the closure device 950 is in the form of a suture passing through or around (e.g., on an outwardly-facing side of) the secondary implantable device 920. The closure device 950 may include a pull end 952 arranged and configured to be pulled (e.g., by a tool 960) to activate, such as to cinch, the closure device 950 to shift the secondary implantable device 920 to an occlusion configuration as illustrated in FIG. 9B.

The pull end 952 may be configured to move in only an occluding direction (causing the secondary implantable device 920 to shift into an occlusion configuration). In the example of an embodiment illustrated in FIG. 10, the pull end 952′ of the closure device 950′ extends through a catch loop 954′ and includes a series of enlarged regions 956′ which can pass through the catch loop 954′ to tighten or cinch the closure device 950. The enlarged regions 956′ resist reverse movement of the pull end 952′ which would allow loosening of the closure device 950″ (shifting the secondary implantable device 920 to an open configuration, such as illustrated in FIG. 9A), and thus hold the secondary implantable device 920 in a selected occlusion configuration. In another example of an embodiment, as illustrated in FIG. 11, the pull end 952″ of the closure device 950″ extends through a catch loop 954″ and includes a series of quills 956″ which can pass through the catch loop 954″ to tighten or cinch the closure device 950″ but which do not permit reverse movement of the pull end 952″ (which would allow loosening of the closure device 950″, shifting the secondary implantable device 920 to an open configuration, such as illustrated in FIG. 9A).

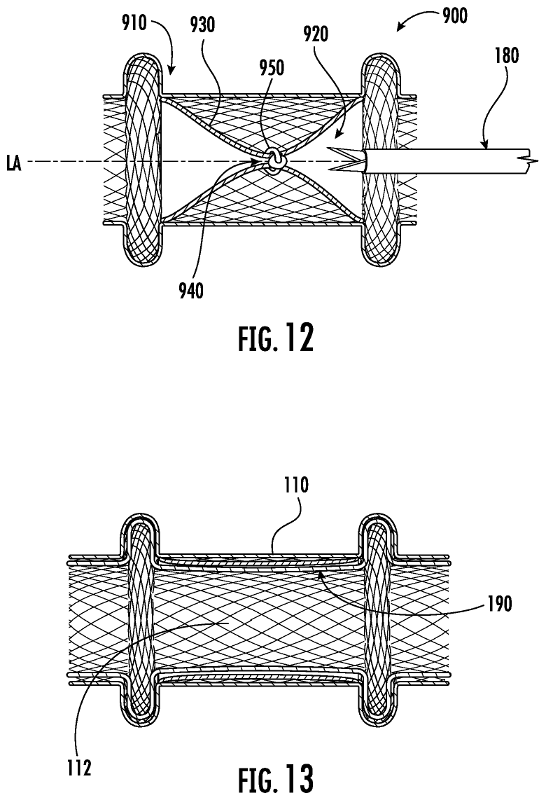

In accordance with various principles of the present disclosure, a secondary implantable device such as disclosed herein may be selectively removable from its position within a primary implantable device such as disclosed herein. In some embodiments, the primary implantable device may be removable as well. In the example of an embodiment of an occlusion device 100 illustrated in FIG. 1, an extension or lip 160 may extend axially from a retention member (such as a proximal retention member) of a primary implantable device and/or a secondary implantable device in a direction axially away from the saddle region 104 of the occlusion device 100. Additionally or alternatively, a removal device 170 may be provided along an end (such as a proximal end 103) of the occlusion device 100 to facilitate removal of the secondary implantable device and optionally also the primary implantable device. In an embodiment of an occlusion device in which the secondary implantable device 920 is in the form of a liner 930 (such as in the example of an embodiment illustrated in FIG. 9A and FIG. 9B), the secondary implantable device 920 may be cut such as by an endoscopic scissor or other cutting tool 180, as illustrated in FIG. 12, and removed to allow passage of materials to resume through the lumen through the occlusion device. To facilitate removal of a primary implantable device formed in accordance with various principles of the present disclosure to anchor an occlusion device to the deployment site, a removal device 190, such as a stent and/or a pressure necrosis device as illustrated in FIG. 13, may be positioned within the lumen 112 of the primary implantable device 110 and expanded radially outwardly to exert pressure on the primary implantable device 110 to cause tissue necrosis allowing removal of the primary implantable device 110 in a manner known to those of ordinary skill in the art.

In view of the above, it should be understood that the various examples of embodiments illustrated in the accompanying figures have several separate and independent features, which each, at least alone, has unique benefits which are desirable for, yet not critical to, the presently disclosed occlusion device. It will be appreciated that various features of the examples of embodiments of occlusion devices described above may be used advantageously and independently of other features. As noted above, occlusion devices may be formed in accordance with various principles of the present disclosure with various combinations of features described herein. That is, any of the features described herein can be mixed and matched to create hybrid designs, and such hybrid designs are within the scope of the present disclosure. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that various aspects of the above disclosure may be applied in other passages within the body to reduce flow through such passage. Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An occlusion device comprising:
a primary implantable device defining a lumen therethrough and configured to anchor with respect to tissue of a body passage;
a secondary implantable device operatively engaged with said primary implantable device; and
an occlusion structure operatively associated with said secondary implantable device and extending across and occluding the lumen of said primary implantable device.

2. The occlusion device of claim 1, wherein said primary implantable device is a stent with at least a portion thereof configured to promote tissue ingrowth.

3. The occlusion device of claim 1, wherein:
said secondary implantable device is a stent extending through at least a part of the lumen of said primary implantable device;
said primary implantable device has a saddle region with a proximal end and a distal end, and at least one retention member extending radially outwardly from the proximal end of said saddle region; and
said secondary implantable device has a proximal retention member configured to operatively engage with a proximal retention member of said primary implantable device to be anchored with respect to the body passage.

4. The occlusion device of claim 3, wherein said occlusion device is a material capable of preventing flow of materials therethrough and is operatively associated with said proximal retention member of said secondary implantable device to extend across the lumen of said primary implantable device.

5. The occlusion device of claim 3, wherein said proximal retention member of said secondary implantable device fits within a retention space defined between walls of a proximal retention member of said primary implantable device.

6. The occlusion device of claim 3, wherein said proximal retention member of said secondary implantable device fits over an exterior portion of a proximal retention member of said primary implantable device.

7. The occlusion device of claim 3, wherein:
said primary implantable device has at least one retention member extending from each end of said saddle region; and
said secondary implantable device has a saddle region with a proximal end and a distal end, said proximal secondary implantable device retention member extending from the proximal end of said saddle region, and said secondary implantable device further including a distal retention member extending from the distal end of said secondary implantable device saddle region.

8. The occlusion device of claim 2, wherein said secondary implantable device is a liner formed within the lumen of said primary implantable device and shiftable into an occlusion configuration defining said occlusion structure across the lumen of said primary implantable device.

9. The occlusion device of claim 1, wherein said occlusion device comprises an occlusive material capable of preventing flow of materials therethrough.

10. The occlusion device of claim 1, wherein said secondary implantable device is selectively removable from said primary implantable device.

11. A method of forming an occlusion device, said method comprising:
engaging a secondary implantable device within a lumen of a primary implantable device configured to anchor with respect to tissue at a deployment site; and
extending an occlusion structure of the secondary implantable device across the lumen of the primary implantable device to occlude flow therethrough.

12. The method of claim 11, wherein the primary implantable device is a stent with uncoated regions configured to encourage tissue ingrowth, said method further comprising deploying the primary implantable device and allowing tissue ingrowth with respect to the primary implantable device before engaging the secondary implantable device within the lumen of the primary implantable device.

13. The method of claim 11, wherein the occlusion structure comprises a material inhibiting tissue ingrowth, said method further comprising extending the occlusion structure in a direction transverse to the longitudinal axis of the primary implantable device to occlude the lumen thereof.

14. The method of claim 13, wherein the primary implantable device is a stent with a saddle region and at least one retention member extending radially outwardly from the saddle region and transverse to the longitudinal axis of the primary implantable device, said method further comprising engaging a retention member of the secondary implantable device with a retention member of the primary implantable device to extend an occlusion structure operatively associated with the retention member of the secondary implantable device across the lumen of the primary implantable device.

15. The method of claim 13, wherein the primary implantable device is a stent with a saddle region defining the primary implantable device lumen therethrough, and the secondary implantable device includes a liner extending through the primary implantable device lumen, said method further comprising extending at least a portion of the liner across the primary implantable device lumen to occlude the primary implantable device lumen.

16. A method of occluding a body passage, said method comprising:
extending a primary implantable device across said body passage to anchor with respect to tissue of the body passage;
securing a secondary implantable device with respect to the primary implantable device; and
extending an occlusion device operatively associated with the secondary implantable device across a lumen extending through the primary implantable device to occlude the lumen.

17. The method of claim 16, wherein securing a secondary implantable device further comprises securing the secondary implantable device with respect to the primary implantable device after sufficient time has elapsed to allow the primary implantable device to be anchored with respect to the tissue of the body passage.

18. The method of claim 17, wherein the sufficient time allows for tissue ingrowth with respect to the primary implantable device to anchor the primary implantable device with respect to the body passage.

19. The method of claim 16, wherein securing the secondary implantable device with respect to the primary implantable device comprises operatively engaging a retention member of the secondary implantable device with a corresponding retention member of the primary implantable device.

20. The method of claim 16, wherein extending the occlusion device comprises shifting a secondary implantable device in the form of a liner within the lumen of the primary implantable device into an occlusion configuration.
